(19) United States
(12) Patent Application Publication
(10) Pub. No.: US 2008/0124292 A1
(43) Pub. Date: May 29, 2008

(54) COMPOSITION CONTAINING AN AMINO ACID COMPOUND

(75) Inventors: Nathalie Collin, Sceaux (FR); Olivier Raineau, Paris (FR); Stephane Arditty, Ballainvilliers (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/938,825

(22) Filed: Nov. 13, 2007

Related U.S. Application Data
(60) Provisional application No. 60/868,754, filed on Dec. 6, 2006.
(60) Provisional application No. 60/868,743, filed on Dec. 6, 2006.
(60) Provisional application No. 60/865,894, filed on Nov. 15, 2006.
(60) Provisional application No. 60/865,864, filed on Nov. 15, 2006.

(30) Foreign Application Priority Data
Nov. 10, 2006 (FR) 06 54825
Nov. 10, 2006 (FR) 06 54828
Nov. 10, 2006 (FR) 06 54831
Nov. 10, 2006 (FR) 06 54840

(51) Int. Cl. A61K 8/42

(52) U.S. Cl. 424/70.7; 424/70.22

(57) ABSTRACT

Composition preferably useful for coating the eyelashes containing an aqueous phase and an emulsifying system which has, as the main surfactant system, at least one surfactant chosen from:
the combination of at least one C₁₆-C₃₀ fatty acid and of at least one basic amino acid,
at least one glutamic acid compound and/or one of its salts,
at least one sarcosine compound of formula:

CH₃—N(R)—CH₂—COOH
in which R is an acyl group O═CR′, R′ being a saturated or unsaturated, linear or branched, hydrocarbon chain comprising from 10 to 30 carbon atoms,
or one of its cosmetically acceptable salts,
a glycine compound and/or a salt of the compound, and their mixtures.

### REFERENCE TO PRIOR APPLICATIONS

This application claims priority to U.S. provisional applications:
60/865,864 filed Nov. 15, 2006,
60/865,894 filed Nov. 15, 2006,
60/868,743 filed Dec. 6, 2006, and
60/868,754 filed Dec. 6, 2006,
   and to French patent applications:
06 54825 filed Nov. 10, 2006,
06 54828 filed Nov. 10, 2006,
06 54831 filed Nov. 10, 2006, and
06 54840 filed Nov. 10, 2006,
   all incorporated herein by reference.

### FIELD OF THE INVENTION

The present patent application relates to compositions containing at least one amino acid compound, and to the filed of making up or caring for the eyelashes or mascaras.

Several advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Compositions for coating the eyelashes, such as mascaras, are generally make-up compositions, compositions to be applied to a make-up (also known as top coat) or also compositions for the cosmetic care of the eyelashes.

Mascaras are prepared in particular according to two types of formulation: water-based mascaras, known as cream mascaras, in the form of a dispersion of waxes in water; anhydrous mascaras or mascaras with a low water content, known as waterproof mascaras, in the form of dispersions of waxes in organic solvents.

In a preferred embodiment the present patent application relates more specifically to water-based mascaras.

The application of mascara makes it possible to increase the volume of the eyelashes and consequently to increase the intensity of the gaze. There exist numerous thickening or volumizing mascaras for doing this, the principle of which consists in depositing as much material as possible on the eyelashes so as to obtain a volumizing (or charging) effect.

It is in particular through the amount of solid particles (in particular waxes, which make it possible to structure the composition) that the specific features of use desired for the compositions can be adjusted, such as, for example, their fluidity or consistency, and also their thickening power (also known as charging or make-up power).

These solid particles are dispersed in the cream mascara using a surfactant system.

The conventional emulsifiers or emulsifying systems include:
cetyl phosphate; however, the use of cetyl phosphate alone results in agglomeration of the pigments and in a coarse dispersion of the waxes;
this frequently resulting in a “grey” and nonblack mascara;
emulsifying systems based on steareth-20 and on steareth-2; however, the use of these systems results in highly fluid mascaras, the consistency of which is not satisfactory for a volumizing mascara;
emulsifying systems based on triethanolamine stearate.

### SUMMARY OF THE INVENTION

One problem addressed in the present patent application is that of providing a mascara in which not only the waxes but also the pigments are homogeneously dispersed, the mascara exhibiting a texture which is sufficiently thick to obtain a volumizing charging deposited layer on the eyelashes and exhibiting a satisfactory consistency which makes possible ready application to the eyelashes and a smooth and homogeneous deposited layer.

The inventors of the present patent application have, surprisingly and unexpectedly, solved this problem using an emulsifying system comprising at least one compound chosen from specific amino acid compounds.

The inventors of the present patent application discovered that the emulsifying system defined in the present patent application provides good dispersion of the pigments and/or waxes; this dispersion is of the quality of those obtained with emulsifying systems based on triethanolamine stearate. This composition makes it possible to obtain a charging make-up for the eyelashes and a smooth and homogeneous deposited layer on the fibres.

The composition according to the invention can exhibit a viscosity ranging for example from 1 to 60 Pa·s, preferably from 1.5 to 50 Pa·s, better still from 2 to 40 Pa·s and even better still from 3 to 30 Pa·s.

The viscosity of the composition is measured at 25° C. using a Rheomat 180 (Lamy) equipped with an MS-R1, MS-R2, MS-R3, MS-R4 or MS-R5 spindle, chosen according to the consistency of the composition, rotating at a rotational speed of 200 rev/min. The measurement is taken after rotating for 10 min.

The compositions in accordance with the invention can have a viscoelastic behaviour.

Generally, a material is said to be viscoelastic when, under the effect of shearing, it has both the characteristics of an elastic material, that is to say capable of storing energy, and the characteristics of a viscous material, that is to say capable of dissipating energy.

The viscoelastic behaviour of the compositions in accordance with the invention can be more particularly characterized by its rigidity modulus G. This parameter is defined in particular in the work “Initiation à la rhéologie” [Introduction to Rheology], G. Couarraze and J. L. Grossiord, 2^{nd} edition, 1991, published by Lavoisier-Tec 1 Doc.

The measurements are carried out on an RS 600 controlled-stress rheometer from ThermoRheo equipped with a thermostatically controlled bath and with a stainless steel rotor of cone/plate geometry, with a diameter of 35 mm and an angle of 2°. The two surfaces are “sanded” to limit phenomena of sliding at the walls.

The measurements are carried out at 25° C.±1° C.

The dynamic measurements are carried out while applying a harmonic variation of the stress. In these experiments, the amplitudes of the shear stress (recorded as τ) and of the shear strain (recorded as γ) are low, so as to remain within the limits of the linear viscoelastic region of the composition (conditions which make it possible to evaluate the Theological characteristics of the composition at rest).

The linear viscoelastic region is generally defined by the fact that the response of the material (i.e. the strain) is at any moment directly proportional to the value of the force applied (i.e. the stress). In this region, the stresses applied are low and the material undergoes strains without modifying its microscopic structure. Under these conditions, the material is studied “at rest” and nondestructively.

The composition is subjected to harmonic shearing according to a stress τ(t) varying sinusoidally according to an angular frequency ω (ω=2πν), ν being the frequency of the shearing applied. The composition thus sheared is subjected to a stress τ(t) and responds according to a strain γ(t) corresponding to microstrains for which the rigidity modulus varies little as a function of the stress imposed.

The stress τ(t) and the strain γ(t) are defined respectively by the following relationships:

τ(t)=τ₀ cos(ω·t) γ(t)=γ₀ cos(ω·t−δ)

τ₀ being the maximum amplitude of the stress and γ₀ being the maximum amplitude of the strain. The elasticity δ is the phase angle between the stress and the strain.

The measurements are carried out at a frequency of 1 Hz (ν=1 Hz).

Increasing stresses are applied to the sample, starting from an initial stress equal to 0.01 Pa, to arrive at a final stress of 1000 Pa, the stresses only being applied once.

The change in the rigidity modulus G (corresponding to the ratio of τ₀ to γ₀) and in the elasticity δ (corresponding to the phase angle of the stress applied with respect to the strain measured) is thus measured as a function of the stress τ(t) applied.

The strain of the composition for the stress zone in which the variation in the rigidity modulus G and in the elasticity δ is less than 7% (microstrain zone) is measured in particular and thus the “plateaux” Gp and δp parameters are determined.

The composition exhibits, for example, a plateau rigidity modulus Gp of greater than or equal to 10 Pa, preferably greater than or equal to 50 Pa, which can range up to 10⁶ Pa and better still up to 5×10⁵ Pa.

A first subject-matter of the present patent application is a composition preferably useful for coating the eyelashes comprising an aqueous phase and an emulsifying system such that the emulsifying system comprises at least one compound chosen from:
the combination of at least one C₁₆-C₃₀ fatty acid and of at least one basic amino acid,
at least one glutamic acid compound and/or one of its salts,
at least one sarcosine compound of formula:

CH₃—N(R)—CH₂—COOH
in which R is an acyl group O═CR′, R′ being a saturated or unsaturated, linear or branched, hydrocarbon chain comprising from 10 to 30 carbon atoms,
or one of its cosmetically acceptable salts,
a glycine compound and/or a salt of the compound, and their mixtures,
   the combination of C₁₆-C₃₀ fatty acid and of basic amino acid or the glutamic acid compound and/or its salt or the sarcosine compound and/or its salt or the glycine compound and/or its salt constituting the main surfactant system of the composition.

The term “main surfactant system” is understood to mean a system which, in its absence, does not result in the formation of a stable composition.

The term “stable” is understood to mean a composition which, after having been placed in an oven at 45° C. for two months, does not exhibit, after returning to ambient temperature, grains perceptible to the touch when a fine layer of the composition is sheared between the fingers.

A second subject-matter of the present patent application is a method for making up or for the nontherapeutic care of the eyelashes comprising the application, to the eyelashes, of the composition according to the present patent application.

A third subject-matter of the present patent application is the uses of the composition according to the present patent application, in particular the use of this composition for obtaining a homogeneous and/or volumizing make-up of the eyelashes.

Other characteristics, properties and advantages of the present invention will become more clearly apparent on reading the description and examples which follow.

### Emulsifying System

The compound chosen from the combination of at least one C₁₆-C₃₀ fatty acid and of at least one basic amino acid, the glutamic acid compound and/or one of its salts, the sarcosine compound or its salts mentioned above and the glycine compound and/or its salts can represent for example from 0.1 to 20% by weight, preferably from 0.5 to 12% by weight, with respect to the total weight of the composition.

a) C₁₆-C₃₀ Fatty Acid and Basic Amino Acid

The C₁₆-C₃₀ fatty acid or acids which can be used in the compositions according to the present application is/are preferably chosen from saturated or unsaturated fatty acids comprising from 16 to 30 carbon atoms and their mixtures. Use is preferably made of a saturated fatty acid advantageously comprising from 16 to 20 carbon atoms. More preferably, it is stearic acid.

Use may advantageously be made of a 50/50 mixture of C₁₆-C₁₈ fatty acids.

According to one embodiment, the content of C₁₆-C₃₀ fatty acid(s) ranges from 0.1 to 20% by weight, preferably from 0.5 to 12% by weight, with respect to the total weight of the composition.

The basic amino acid(s) which can be used in the compositions according to the present patent application is/are preferably chosen from lysine, arginine and histidine; preferably, it is lysine.

According to one embodiment, the content of basic amino acid(s) ranges for example from 0.1 to 20% by weight, with respect to the total weight of the composition, preferably from 0.5 to 10% by weight.

Preferably, the emulsifying system comprising the combination of at least one C₁₆-C₃₀ fatty acid and of at least one basic amino acid constitutes the main surfactant system of the composition.

Advantageously, the emulsifying system comprising the combination of at least one C₁₆-C₃₀ fatty acid and of at least one basic amino acid constitutes the sole surfactant system of the composition.

The term “sole” is understood to mean that any optional additional surfactant system is present in a content not exceeding 1% and preferably not exceeding 0.5%. More preferably, the term “sole” denotes the complete absence of any other surfactant system.

b) Glutamic Acid Compound

The glutamic acid salt or compound can, for example, be chosen from acyl glutamic acids (INCI name: acyl glutamic acid), their salts (glutamates) and their mixtures, preferably from acyl glutamic acids having an acyl group comprising from 10 to 30 carbon atoms, preferably from 12 to 22 carbon atoms, such as, for example, lauroyl glutamic acid, myristoyl glutamic acid, palmitoyl glutamic acid, stearoyl glutamic acid, behenoyl glutamic acid, olivoyl glutamic acid or cocoyl glutamic acid, and the salts of alkali metals, such as Na, Li or K, preferably Na or K, the salts of alkaline earth metals, such as Mg, or the ammonium salts of the acids.

Mention may in particular be made of the compounds carrying the INCI name lauroyl glutamic acid, cocoyl glutamic acid, sodium stearoyl glutamate, potassium lauroyl glutamate, potassium cocoyl glutamate, sodium olivoyl glutamate and their mixtures.

Such compounds are sold under the name Amisoft by Ajinomoto and in particular under the references Amisoft CA, Amisoft LA, Amisoft HS 11 PF, Amisoft MK-11, Amisoft LK-11 and Amisoft CK-11 or are also sold by Keminova Italiana SRL.

Mention may also be made, as salt of glutamic acid compound, of disodium hydrogenated tallow glutamate, such as that sold under the reference Amisoft HS-21 by Ajinomoto.

Mention may also be made of commercial mixtures of surfactants comprising at least one glutamic acid compound or a salt of the compound, such as, for example, the mixture of acyl glutamate salts, such as Amisoft LS-22, sold by Ajinomoto.

The glutamic acid compounds and their salts can be present in the composition in a content ranging for example from 0.1 to 20% by weight, preferably from 0.5 to 15% by weight and better still from 1 to 10% by weight, with respect to the total weight of the composition.

According to one embodiment, the glutamic acid compound or its salt is present in a content of greater than or equal to 1% by weight, with respect to the total weight of the composition.

According to one embodiment, the glutamic acid compounds and their salts can be present in the composition in a content ranging for example from 1 to 20% by weight, preferably from 1.5 to 15% by weight and better still from 2 to 10% by weight, with respect to the total weight of the composition.

According to one embodiment, the glutamic acid compounds and their salts constitute the main surfactant system (as defined above) of the composition.

Advantageously, the glutamic acid compounds and their salts constitute the sole surfactant system (as defined above) of the composition.

c) Sarcosine Compound

The sarcosine compound(s) which can be used in the compositions according to the present patent application is/are preferably chosen from the sarcosine compounds of formula:

CH₃—N(R)—CH₂—COOH

in which R is an acyl group O═CR′, R′ being a saturated or unsaturated, linear or branched, hydrocarbon chain comprising from 10 to 30 carbon atoms, preferably from 12 to 22 carbon atoms. R can, for example, be a lauroyl, myristoyl, palmitoyl, oleoyl or stearoyl group and their mixtures.

Use will advantageously be made of myristoyl sarcosine compounds, palmitoyl sarcosine compounds, oleoyl sarcosine compounds or stearoyl sarcosine compounds, preferably stearoyl and palmitoyl sarcosine compounds, or their cosmetically acceptable salts (sarcosinates).

Mention may in particular be made of sodium palmitoyl sarcosinate, magnesium palmitoyl sarcosinate, myristoyl sarcosine, stearoyl sarcosine and their mixtures.

These compounds are, for example, sold by Croda under the name Crodasinic MS or Crodasinic O, or by Nikko Chemicals under the name Nikkol sarcosinate MN, Nikkol sarcosinate PN or Nikkol sarcosinate OH, or by Seppic under the name Oramix O, or by Kawaken Fine Chemicals under the name Soypon O or Soypon S.

Use may also be made of mixtures of commercial surfactants formed of sarcosine compounds, such as Crodasinic SM, sold by Croda.

These sarcosine compounds can be used alone or as mixtures in all proportions.

The content of sarcosine compound(s) preferably ranges from 0.1 to 20% by weight, more preferably from 0.5 to 15% by weight and even more preferably from 1 to 10% by weight, with respect to the total weight of the composition.

Preferably, the sarcosine compound(s) constitute(s) the main surfactant system of the composition.

Advantageously, the sarcosine compound(s) constitute(s) the sole surfactant system of the composition.

d) Glycine Compound

The glycine compound or its salt can be chosen from glycine salts (or glycinates) and in particular from:

a) Acyl glycinates of formula (I):

R—HNCH₂COOX (I)

in which R represents an acyl group R′C═O, with R′, which represents a saturated or unsaturated, linear or branched, hydrocarbon chain, preferably comprising from 10 to 30 carbon atoms, preferably from 12 to 22 carbon atoms, preferably from 14 to 22 carbon atoms and better still from 16 to 20 carbon atoms, and

X represents a cation chosen, for example, from ions of alkali metals, such as Na, Li or K, preferably Na or K, ions of alkaline earth metals, such as Mg, ammonium groups and their mixtures.

The acyl group can be chosen in particular from the lauroyl, myristoyl, behenoyl, palmitoyl, stearoyl, isostearoyl, olivoyl, cocoyl or oleoyl groups and their mixtures.

Preferably, R is a cocoyl group.

b) Glycinates of following formula (II):

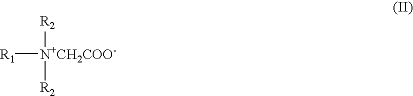

in which
R₁ represents a saturated or unsaturated, linear or branched, hydrocarbon chain comprising from 10 to 30 carbon atoms, preferably from 12 to 22 carbon atoms and better still from 16 to 20 carbon atoms;

R₁ is advantageously chosen from the lauryl, myristyl, palmityl, stearyl, cetyl, cetearyl or oleyl groups and their mixtures and preferably from the stearyl and oleyl groups,
the R₂ groups, which are identical or different, represent an R₃OH group, R₃ being an alkyl group comprising from 2 to 10 carbon atoms, preferably from 2 to 5 carbon atoms.

Mention may be made, as compound of formula (I), for example, of the compounds carrying the INCI name sodium cocoyl glycinate, such as, for example, Amilite GCS-12, sold by Ajinomoto, or potassium cocoyl glycinate, such as, for example, Amilite GCK-12 from Ajinomoto.

Use may be made, as compounds of formula (II), of dihydroxyethyl oleyl glycinate or dihydroxyethyl stearyl glycinate.

The glycine compounds and their salts can be present in the composition in a content ranging for example from 0.1 to 20% by weight, preferably from 0.5 to 15% by weight and better still from 1 to 10% by weight, with respect to the total weight of the composition.

According to one embodiment, the glycine compound or compounds and their salts constitute the main surfactant system of the composition.

Advantageously, the glycine compound or compounds and their salts constitute the sole surfactant system of the composition.

The composition according to the invention comprises, of course, a physiologically acceptable medium. The term “physiologically acceptable compound or medium” is understood to mean, within the meaning of the present patent application, a compound or medium, the use of which is compatible with application to the eyelashes.

### Aqueous Phase

The composition according to the invention comprises an aqueous phase which can form the continuous phase of the composition.

The term “composition comprising an aqueous continuous phase” is understood to mean that the composition exhibits a conductivity, measured at 25° C., of greater than or equal to 23 μS/cm (microSiemens/cm), the conductivity being measured, for example, using an MPC227 conductivity meter from Mettler Toledo and an Inlab730 conductivity measurement cell. The measurement cell is immersed in the composition, so as to remove the air bubbles liable to be formed between the two electrodes of the cell. The conductivity is read as soon as the value of the conductivity meter has stabilized. A mean is determined over at least 3 successive measurements.

The aqueous phase comprises water and/or at least one water-soluble solvent.

The term “water-soluble solvent” denotes, in the present invention, a compound which is liquid at ambient temperature and which is miscible with water (miscibility in water of greater than 50% by weight at 25° C. and atmospheric pressure).

The water-soluble solvents which can be used in the compositions according to the invention can in addition be volatile.

Mention may in particular be made, among the water-soluble solvents which can be used in the compositions in accordance with the invention, of lower monoalcohols having from 1 to 5 carbon atoms, such as ethanol and isopropanol, or glycols having from 2 to 8 carbon atoms, such as ethylene glycol, propylene glycol, 1,3-butylene glycol and dipropylene glycol.

The aqueous phase (water and optionally the water-miscible solvent) is generally present in the composition according to the present patent application in a content ranging for example from 1% to 95% by weight, with respect to the total weight of the composition, preferably ranging for example from 3% to 80% by weight and preferentially ranging for example from 5% to 60% by weight.

The emulsifying system can moreover comprise at least one additional surface-active agent appropriately chosen in order to obtain a wax-in-water or oil-in-water emulsion.

Use may in particular be made of an emulsifier having, at 25° C., an HLB balance (hydrophilic-lipophilic balance), within the Griffin meaning, of greater than or equal to 8.

These additional surface-active agents can be chosen from nonionic, anionic, cationic or amphoteric surface-active agents or surface-active emulsifiers. Reference may be made to the document “Encyclopedia of Chemical Technology, Kirk-Othmer”, volume 22, pp. 333-432, 3^{rd} edition, 1979, Wiley, for the definition of the properties and functions (emulsifying) of surfactants, in particular pp. 347-377 of this reference for the anionic, amphoteric and nonionic surfactants.

These additional surfactants can preferably be chosen from:
a) nonionic surface-active agents with an HLB of greater than or equal to 8 at 25° C., used alone or as a mixture; mention may in particular be made of:
   oxyethylenated and/or oxypropylenated ethers (which can comprise from 1 to 150 oxyethylene and/or oxypropylene groups) of glycerol;
   oxyethylenated and/or oxypropylenated ethers (which can comprise from 20 to 1000 oxyethylene and/or oxypropylene groups) of fatty alcohols (in particular of C₈-C₂₄ and preferably C₁₂-C₁₈ alcohols), such as the oxyethylenated ether of cetearyl alcohol comprising 30 oxyethylene groups (CTFA name “Ceteareth-30”), the oxyethylenated ether of stearyl alcohol comprising 20 oxyethylene groups (CTFA name “Steareth-20”), such as the Brij 78 sold by Uniqema, and the oxyethylenated ether of the mixture of C₁₂-C₁₅ fatty alcohols comprising 7 oxyethylene groups (CTFA name “C12-15 Pareth-7”), sold under the name Neodol 25-7 by Shell Chemicals,
   esters of fatty acid (in particular of C₈-C₂₄ and preferably C₁₆-C₂₂ acid) and of polyethylene glycol (which can comprise from 1 to 150 ethylene glycol units), such as PEG-50 stearate and PEG-40 monostearate, sold under the name Myrj 52P® by ICI Uniqema,
   esters of fatty acid (in particular of C₈-C₂₄ and preferably C₁₆-C₂₂ acid) and of oxyethylenated and/or oxypropylenated glycerol ethers (which can comprise from 1 to 150 oxyethylene and/or oxypropylene groups), such as PEG-200 glyceryl monostearate, sold under the name Simulsol 220 TM® by Seppic; polyethoxylated glyceryl stearate comprising 30 ethylene oxide groups, such as the product Tagat S® sold by Goldschmidt, polyethoxylated glyceryl oleate comprising 30 ethylene oxide groups, such as the product Tagat O® sold by Goldschmidt, polyethoxylated glyceryl cocoate comprising 30 ethylene oxide groups, such as the product Varionic LI 13® sold by Sherex, polyethoxylated glyceryl isostearate comprising 30 ethylene oxide groups, such as the product Tagat L® sold by Goldschmidt, and polyethoxylated glyceryl laurate comprising 30 ethylene oxide groups, such as the product Tagat I® from Goldschmidt,
   esters of fatty acid (in particular of C₈-C₂₄ and preferably C₁₆-C₂₂ acid) and of oxyethylenated and/or oxypropylenated sorbitol ethers (which can comprise from 1 to 150 oxyethylene and/or oxypropylene groups), such as polysorbate 60, sold under the name Tween 60® by Uniqema,
   dimethicone copolyol, such as that sold under the name Q2-5220® by Dow Corning,
   dimethicone copolyol benzoate (Finsolv SLB 101® and 201® from Fintex),
   copolymers of propylene oxide and of ethylene oxide, also known as EO/PO polycondensates,
   and their mixtures.

The EO/PO polycondensates are more particularly copolymers consisting of polyethylene glycol and polypropylene glycol blocks, such as, for example, polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH

in which formula a ranges from 2 to 120 and b ranges from 1 to 100.

The EO/PO polycondensate preferably has a weight-average molecular weight ranging for example from 1000 to 15 000 and better still ranging form 2000 to 13 000. Advantageously, the EO/PO polycondensate has a cloud point, at 10 g/l in distilled water, of greater than or equal to 20° C., preferably of greater than or equal to 60° C. The cloud point is measured according to Standard ISO 1065. Mention may be made, as EO/PO polycondensate which can be used according to the invention, of the polyethylene glycol/polypropylene glycol/polyethylene glycol triblock polycondensates sold under the Synperonic® names, such as Synperonic PE/L44® and Synperonic PE/F127®, by ICI.
b) nonionic surface-active agents with an HLB of less than 8 at 25° C., optionally in combination with one or more nonionic surface-active agents with an HLB of greater than 8 at 25° C., such as mentioned above, such as:
   esters and ethers of monosaccharides, such as sucrose stearate, sucrose cocoate, sorbitan stearate and their mixtures, such as Arlatone 2121®, sold by ICI;
   esters of fatty acids (in particular of C₈-C₂₄ and preferably C₁₆-C₂₂ acid) and of polyol, in particular of glycerol or of sorbitol, such as glyceryl stearate, such as the product sold under the name Tegin M® by Goldschmidt, glyceryl laurate, such as the product sold under the name Imwitor 312® by Hüls, polyglyceryl-2 stearate, sorbitan tristearate or glyceryl ricinoleate;
   the cyclomethicone/dimethicone copolyol mixture sold under the name Q2-3225C® by Dow Corning.
   c) anionic surfactants, such as:
   salts of C₁₆-C₃₀ fatty acids, in particular those deriving from amines, such as triethanolamine stearate and/or 2-amino-2-methylpropane-1,3-diol stearate; however, preferably, the composition according to the present patent application does not comprise triethanolamine stearate;
   salts of polyoxyethylenated fatty acids, in particular those deriving from amines or the alkali metal salts, and their mixtures;
   alkyl ether sulphates, such as sodium lauryl ether sulphate;
   isethionates.

The composition according to the invention can also comprise one or more amphoteric surfactants, such as N-acylamino acids, for example N-alkylaminoacetates and disodium cocoamphodiacetate, and amine oxides, such as stearamine oxide, or also silicone surfactants, such as dimethicone copolyol phosphates, for example that sold under the name Pecosil PS 100® by Phoenix Chemical.

According to an alternative form, the cosmetic composition according to the present patent application preferably comprises less than 1% by weight, preferably less than 0.5% by weight, of triethanolamine and better still is devoid of triethanolamine.

According to a preferred alternative form, the cosmetic composition according to the present patent application preferably comprises less than 1% by weight, more preferably less than 0.5% by weight, of triethanolamine stearate and better still is devoid of triethanolamine stearate.

According to the present invention, the additional surface-active agent is not a surfactant system as defined above, given that this additional surface-active agent alone cannot result in the formation of a stable composition, as defined above.

The total amount of surfactants in the composition preferably can range from 0.5 to 20% by weight, preferably from 1 to 10% by weight, with respect to the total weight of the composition.

According to one embodiment, the composition can additionally comprise a cosurfactant which can be chosen from fatty alcohols, preferably comprising from 10 to 30 carbon atoms. The term “fatty alcohol comprising from 10 to 30 carbon atoms” is understood to mean any pure, saturated or unsaturated, branched or unbranched, fatty alcohol comprising from 10 to 30 carbon atoms.

Use is preferably made of a fatty alcohol comprising from 10 to 26 carbon atoms, better still from 10 to 24 carbon atoms and even better still from 14 to 22 carbon atoms.

Mention may in particular be made, as fatty alcohols which can be used in the composition, of lauryl, myristyl, cetyl, stearyl, oleyl, cetearyl (mixture of cetyl alcohol and stearyl alcohol), behenyl or erucyl alcohols and their mixtures. Use is preferably made of cetyl alcohol.

Such fatty alcohols are sold in particular under the name Nafol by Sasol.

The cosurfactant can be present in a content ranging for example from 0.2 to 20% by weight, preferably from 0.3 to 10% by weight, with respect to the total weight of the composition.

In the composition in accordance with the invention, the total content of surface-active agents can range for example from 0.1 to 30% by weight, with respect to the total weight of the composition, preferably from 1 to 20% by weight and better still from 2 to 15% by weight.

### Wax(es)

The composition according to the present patent application advantageously comprises at least one wax.

The wax can be present in a content ranging for example from 0.1 to 50% by weight, with respect to the total weight of the composition, better still from 1 to 40% by weight and even better still from 5 to 30% by weight.

According to one embodiment, the wax is present in a content of greater than or equal to 5% by weight, with respect to the total weight of the composition, preferably in a content of greater than or equal to 10% by weight and better still of greater than or equal to 15% by weight.

The term “wax” is understood to mean, within the meaning of the present invention, a lipophilic compound which is solid at ambient temperature (25° C.), which exhibits a reversible solid/liquid change in state and which has a melting point of greater than or equal to 30° C. which can range up to 120° C.

The melting point of the wax can be measured using a differential scanning calorimeter (DSC), for example the calorimeter sold under the name DSC 30 by Metler.

The waxes can be hydrocarbon, fluorinated and/or silicone waxes and can be of vegetable, mineral, animal and/or synthetic origin. In particular, the waxes exhibit a melting point of greater than 25° C. and better still of greater than 45° C.

The wax can be present in a content ranging for example from 0.1 to 50% by weight, with respect to the total weight of the composition, better still from 1 to 40% by weight and even better still from 5 to 30% by weight.

Use may in particular be made of hydrocarbon waxes, such as beeswax, lanolin wax and Chinese insect waxes; rice wax, carnauba wax, candelilla wax, ouricury wax, esparto wax, cork fibre wax, sugarcane wax, Japan wax and sumac wax; montan wax, microcrystalline waxes, paraffin waxes and ozokerite; polyethylene waxes, waxes obtained by the Fischer-Tropsch synthesis and waxy copolymers, and also their esters.

Mention may also be made of the waxes obtained by catalytic hydrogenation of animal or vegetable oils having linear or branched C₈-C₃₂ fatty chains.

Mention may in particular be made, among these, of hydrogenated jojoba oil, isomerized jojoba oil, such as the trans-isomerized partially hydrogenated jojoba oil manufactured or sold by Desert Whale under the commercial reference Iso-Jojoba-50®, hydrogenated sunflower oil, hydrogenated castor oil, hydrogenated coconut oil, hydrogenated lanolin oil, di(1,1,1-trimethylolpropane) tetrastearate, sold under the name “Hest 2T-4S” by Heterene, or di(1,1,1-trimethylolpropane) tetrabehenate, sold under the name “Hest 2T-4B” by Heterene.

Mention may also be made of silicone waxes, such as alkyl or alkoxy dimethicones having from 16 to 45 carbon atoms, or fluorinated waxes.

Use may also be made of the wax obtained by hydrogenation of olive oil esterified with stearyl alcohol sold under the name “Phytowax Olive 18 L 57” or else of the waxes obtained by hydrogenation of castor oil esterified with cetyl alcohol sold under the names “Phytowax castor 16L64” and “Phytowax castor 22L73” by Sophim. Such waxes are described in Application FR-A-2 792 190.

According to a specific embodiment, the compositions in accordance with the invention can comprise at least one wax known as “tacky wax”, that is to say having a tack of greater than or equal to 0.7 N.s and a hardness of less than or equal to 3.5 MPa.

The use of a tacky wax can in particular make it possible to obtain a cosmetic composition which is easily applied to the eyelashes, which has good attachment to the eyelashes and which results in the formation of a smooth, homogeneous and thickening make-up.

The tacky wax used can in particular have a tack ranging for example from 0.7 N.s to 30 N.s, in particular of greater than or equal to 1 N.s, in particular ranging for example from 1 N.s to 20 N.s, especially of greater than or equal to 2 N.s, in particular ranging for example from 2 N.s to 10 N.s, and especially ranging for example from 2 N.s to 5 N.s.

The tack of the wax is determined by the measurement of the change in the force (compressive force or stretching force) as a function of the time at 20° C. using the texture analyser sold under the name “TA-TX2i®” by Rheo, equipped with a spindle made of acrylic polymer in the shape of a cone forming an angle of 45°.

The measurement protocol is as follows:

The wax is melted at a temperature equal to the melting point of the wax+10° C. The molten wax is cast in a receptacle with a diameter of 25 mm and a depth of 20 mm. The wax is recrystallized at ambient temperature (25° C.) for 24 hours, so that the surface of the wax is flat and smooth, and then the wax is stored at 20° C. for at least 1 hour before measuring the tack.

The spindle of the texture analyser is displaced at the rate of 0.5 mm/s and then penetrates the wax to a penetration depth of 2 mm. When the spindle has penetrated the wax to a depth of 2 mm, the spindle is held stationary for 1 second (corresponding to the relaxation time) and is then withdrawn at the rate of 0.5 mm/s.

During the relaxation time, the force (compressive force) strongly decreases until it becomes zero and then, during the withdrawal of the spindle, the force (stretching force) becomes negative to subsequently again increase towards the value of 0. The tack corresponds to the integral of the curve of the force as a function of the time for the part of the curve corresponding to the negative values of the force (stretching force). The value of the tack is expressed in N.s.

The tacky wax which can be used generally has a hardness of less than or equal to 3.5 MPa, in particular ranging for example from 0.01 MPa to 3.5 MPa, especially ranging for example from 0.05 MPa to 3 MPa, indeed even ranging for example from 0.1 MPa to 2.5 MPa.

The hardness is measured according to the protocol described above.

Use may be made, as tacky wax, of a C₂₀-C₄₀ alkyl (hydroxystearyloxy)stearate (the alkyl group comprising from 20 to 40 carbon atoms), alone or as a mixture, in particular a C₂₀-C₄₀ alkyl 12-(12′-hydroxystearyloxy)stearate, of formula (II):

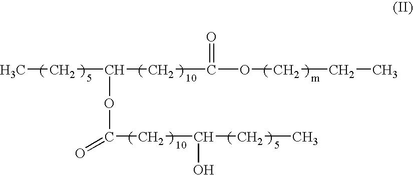

in which m is an integer ranging for example from 18 to 38, or a mixture of compounds of formula (II).

Such a wax is sold in particular under the names “Kester Wax K 82 P®” and “Kester Wax K 80 P®” by Koster Keunen.

The abovementioned waxes generally exhibit a starting melting point of less than 45° C.

Use may also be made of the microcrystalline wax sold under the reference SP18 by Strahl and Pitsch, which exhibits a hardness of approximately 0.46 MPa and a tack value of approximately 1 N.s.

The wax or waxes can be present in the form of an aqueous wax microdispersion. The term “aqueous wax microdispersion” is understood to mean an aqueous dispersion of wax particles in which the size of the wax particles is less than or equal to approximately 1 μm.

Wax microdispersions are stable dispersions of colloidal wax particles and are described in particular in “Microemulsions Theory and Practice”, edited by L. M. Prince, Academic Press (1977), pages 21-32.

In particular, these wax microdispersions can be obtained by melting the wax in the presence of a surfactant and optionally of a portion of the water and then gradually adding hot water with stirring. The intermediate formation of an emulsion of the water-in-oil type, followed by phase inversion, with a microemulsion of oil-in-water type finally being obtained, is observed. On cooling, a stable microdispersion of solid colloidal wax particles is obtained.

The wax microdispersions can also be obtained by stirring the mixture of wax, of surfactant and of water using stirring means, such as ultrasound, a high pressure homogenizer or turbine mixers.

The particles of the wax microdispersion preferably have mean sizes of less than 1 μm (in particular ranging for example from 0.02 μm to 0.99 μm), preferably of less than 0.5 μm (in particular ranging for example from 0.06 μm to 0.5 μm).

These particles are composed essentially of a wax or of a mixture of waxes. However, they can comprise a minor proportion of oily and/or pasty fatty additives, a surfactant and/or a conventional fat-soluble additive/active principle.

The compositions according to the present patent application can also comprise at least one hydrophilic or lipophilic film-forming polymer.

In the present patent application, the term “film-forming polymer” is understood to mean a polymer capable of forming, by itself alone or in the presence of an additional agent which is able to form a film, a macroscopically continuous film which adheres to the eyelashes and preferably a cohesive film and better still a film, the cohesion and the mechanical properties of which are such that the film can be isolable and handleable in isolation, for example when the film is produced by casting on a nonstick surface, such as a Teflon- or silicone-treated surface.

Generally, the content of “film-forming polymer” of the compositions according to the present patent application ranges for example from 0.1 to 40% by weight, preferably from 0.5 to 30% by weight and better still from 1 to 20% by weight, with respect to the total weight of the composition.

The hydrophilic film-forming polymer can be a water-soluble polymer or can be provided in dispersion in an aqueous medium.

Mention may be made, among the film-forming polymers which can be used in the composition of the present invention, of synthetic polymers of radical type or of polycondensate type, polymers of natural origin, and their mixtures.

Mention may be made, as examples of water-soluble film-forming polymers, of:
proteins, such as proteins of plant origin, such as wheat or soya proteins; proteins of animal origin, such as keratins, for example keratin hydrolysates and sulphonic keratins;
cellulose polymers, such as hydroxyethylcellulose, hydroxypropylcellulose, methylcellulose, ethylhydroxyethylcellulose, carboxymethylcellulose and quaternized cellulose compounds;
acrylic polymers or copolymers, such as polyacrylates or polymethacrylates;
vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate, copolymers of vinylpyrrolidone and of caprolactam, or polyvinyl alcohol;
anionic, cationic, amphoteric or nonionic chitin or chitosan polymers;
gums arabic, guar gum, xanthan compounds or karaya gum;
alginates and carrageenans;
glycoaminoglycans, hyaluronic acid and its compounds;
shellac resin, gum sandarac, dammars, elemis or copals;
deoxyribonucleic acid;
mucopolysaccharides, such as chondroitin sulphates;
   and their mixtures.

The film-forming polymer can also be present in the composition in the form of particles in dispersion in an aqueous phase, generally known under the name of latex or pseudolatex. The techniques for preparing these dispersions are well known to a person skilled in the art.

Use may be made, as aqueous film-forming polymer dispersion, of acrylic dispersions, sold under the names Neocryl XK-90®, Neocryl A-1070®, Neocryl A-1090®, Neocryl BT-62®, Neocryl A-1079® and Neocryl A-523® by Avecia-Neoresins, Dow Latex 432® by Dow Chemical, Daitosol 5000 AD® or Daitosol 5000 SJ® by Daito Kasey Kogyo; Syntran 5760® by Interpolymer or Allianz Opt® by Röhm & Haas, or also aqueous dispersions of polyurethane, sold under the names Neorez R-981® and Neorez R-974® by Avecia-Neoresins, Avalure UR-405®, Avalure UR-410®, Avalure UR-425®, Avalure UR-450®, Sancure 875®, Avalure UR-445® and Sancure 2060® by Noveon, Impranil 85® by Bayer or Aquamere H-1511® by Hydromer; sulphopolyesters, sold under the trade name Eastman AQ® by Eastman Chemical Products, vinyl dispersions, such as Mexomer PAM®, aqueous dispersions of poly(vinyl acetate), such as “Vinybran®” from Nisshin Chemical or those sold by Union Carbide, aqueous dispersions of terpolymer of vinylpyrrolidone, dimethylaminopropylmethacrylamide and lauryldimethyl-methacrylamidopropylammonium chloride, such as Styleze W from ISP, aqueous dispersions of polyurethane/polyacrylic hybrid polymers, such as those sold under the references “Hybridur®” by Air Products or “Duromer®” by National Starch, dispersions of core/shell type: for example those sold by Atofina under the Kynar reference (core: fluorinated—shell: acrylic) or also those described in the document U.S. Pat. No. 5,188,899 (core: silica—shell: silicone), and their mixtures.

The lipophilic polymer can be in solution or in dispersion in a nonaqueous solvent phase.

The compositions according to the present patent application can also comprise at least one hydrophilic gelling agent; they can be chosen for example from:
homo- or copolymers of acrylic acid or methacrylic acid or their salts and their esters and in particular the products sold under the names Versicol F® or Versicol K® by Allied Colloid, Ultrahold 8® by Ciba-Geigy, polyacrylic acids of Synthalen K type,
copolymers of acrylic acid and of acrylamide, sold in the form of their sodium salt under the Reten® names by Hercules, sodium salts of polyhydroxy-carboxylic acids, sold under the name Hydagen F® by Henkel,
copolymers of polyacrylic acids and of alkyl acrylates of Pemulen type,
AMPS (polyacrylamidomethylpropanesulphonic acid partially neutralized with aqueous ammonia and highly crosslinked), sold by Clariant,
AMPS/acrylamide copolymers of Sepigel® or Simulgel® type, sold by Seppic,
copolymers of AMPS and of alkyl methacrylates which are polyoxyethylenated (crosslinked or noncrosslinked),
associative polyurethanes, such as the polymer C₁₆-OE₁₂₀-C₁₆ from Servo Delden (sold under the name SER AD FX1100, molecule comprising a urethane functional group and with a weight-average molecular weight of 1300), OE being an oxyethylene unit, Rheolate 205 comprising a urea functional group, sold by Rheox, or also Rheolate 208 or 204 (these polymers being sold in the pure form), or DW 1206B from Röhm & Haas comprising a C₂₀ alkyl chain and comprising a urethane bond, sold at 20% of active material in water. Use may also be made of solutions or dispersions of these associative polyurethanes, in particular in water or in an aqueous/alcoholic medium. Mention may be made, by way of example of such polymers, of SER AD fx1010, SER AD FX1035 and SER AD 1070 from Servo Delden and Rheolate 255, Rheolate 278 and Rheolate 244, sold by Rheox. Use may also be made of the products DW 1206F and DW 1206J, and also Acrysol RM 184 or Acrysol 44, from Röhm & Haas, or alternatively Borchigel LW 44 from Borchers,
and their mixtures.

Some water-soluble film-forming polymers mentioned above can also act as water-soluble gelling agent.

The hydrophilic gelling agents can be present in the compositions according to the invention in a content ranging for example from 0.05 to 40% by weight, with respect to the total weight of the composition, preferably from 0.1 to 20% by weight and better still from 0.5 to 15% by weight.

The compositions according to the present patent application can also comprise at least one or more oils or organic solvents.

The term “oil or organic solvent” is understood to mean a nonaqueous body which is liquid at ambient temperature and atmospheric pressure. The oil can be volatile or nonvolatile.

The term “volatile oil or organic solvent” is understood to mean, within the meaning of the invention, any nonaqueous medium capable of evaporating on contact with keratinous substances in less than one hour at ambient temperature and atmospheric pressure. The volatile organic solvent or solvents and the volatile oils of the invention are volatile cosmetic organic solvents and oils which are liquid at ambient temperature and which have a non-zero vapour pressure, at ambient temperature and atmospheric pressure, ranging for example from 0.13 Pa to 40 000 Pa (10⁻³ to 300 mmHg), in particular ranging for example from 1.3 Pa to 13 000 Pa (0.01 to 100 mmHg) and more particularly ranging for example from 1.3 Pa to 1300 Pa (0.01 to 10 mmHg). The term “nonvolatile oil” is understood to mean an oil which remains on keratinous substances at ambient temperature and atmospheric pressure for at least several hours and which has in particular a vapour pressure of less than 10⁻³ mmHg (0.13 Pa).

The oil can be present in the composition in a content ranging for example from 0.05 to 30% by weight, preferably 0.1 to 15% by weight, with respect to the total weight of the composition. The composition according to the invention can comprise volatile oils and/or nonvolatile oils, and their mixtures.

The volatile oils (or organic solvents) can be hydrocarbon oils, silicone oils, fluorinated oils or their mixtures.

The term “hydrocarbon oil” is understood to mean an oil comprising mainly hydrogen and carbon atoms and optionally oxygen, nitrogen, sulphur or phosphorus atoms. Volatile hydrocarbon oils can be chosen from hydrocarbon oils having from 8 to 16 carbon atoms, in particular branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexa-decane, for example the oils sold under the “Isopars®” or “Permethyls®” trade names, branched C₈-C₁₆ esters, isohexyl neopentanoate, and their mixtures. Other volatile hydrocarbon oils, such as petroleum distillates, in particular those sold under the “Shell Solt®” name by Shell, can also be used.

Use may also be made, as volatile oils, of volatile silicones, such as, for example, volatile linear or cyclic silicone oils, in particular those having a viscosity≦6 centistokes (6×10⁻⁶ m²/s) and having in particular from 3 to 6 silicon atoms, these silicones optionally comprising one or more alkyl or alkoxy groups having 1 or 2 carbon atoms. Mention may in particular be made, as volatile silicone oil which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Use may also be made of volatile organic solvents, in particular fluorinated organic solvents, such as nonafluoromethoxybutane or perfluoromethylcyclopentane.

Each of the compositions in accordance with the invention can also comprise at least one nonvolatile oil or organic solvent which can be chosen in particular from nonvolatile hydrocarbon oils and/or silicone oils and/or fluorinated oils.

Mention may in particular be made, as nonvolatile hydrocarbon oil, of:
hydrocarbon oils of vegetable origin, such as triglycerides composed of esters of fatty acids and of glycerol, the fatty acids of which can have varied chain lengths from C₄ to C₂₄, it being possible for these chains to be linear or branched and saturated or unsaturated; these oils are in particular wheat germ, sunflower, grape seed, sesame, maize, apricot kernel, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; or triglycerides of caprylic/capric acids, such as those sold by Stéarineries Dubois or those sold under the names Miglyol 810®, 812® and 818® by Dynamit Nobel,
synthetic ethers having from 10 to 40 carbon atoms;
linear or branched hydrocarbons of mineral or synthetic origin, such as liquid petrolatum, polydecenes, hydrogenated polyisobutene, such as Parleam oil, squalane, and their mixtures;
synthetic esters, such as the oils of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂ is ≧10, such as, for example, Purcellin oil (cetearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, octanoates, decanoates or ricinoleates of alcohols or of polyalcohols, such as propylene glycol dioctanoate; hydroxylated esters, such as isostearyl lactate or diisostearyl malate; and pentaerythritol esters;
fatty alcohols which are liquid at ambient temperature comprising a branched and/or unsaturated carbon chain having from 12 to 26 carbon atoms, such as octyldodecanol, isostearyl alcohol, oleyl alcohol, 2-hexyldecanol, 2-butyloctanol or 2-undecylpentadecanol;
higher fatty acids, such as oleic acid, linoleic acid or linolenic acid;
and their mixtures.

The nonvolatile silicone oils which can be used in the composition in accordance with the invention can be polydimethylsiloxanes (PDMSs) which are nonvolatile, polydimethylsiloxanes comprising pendent alkyl or alkoxy groups and/or alkyl or alkoxy groups at the end of the silicone chain, groups each having from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

The fluorinated oils which can be used in the compositions in accordance with the invention are in particular fluorosilicone oils, fluorinated polyethers or fluorinated silicones, such as described in the document EP-A-847 752.

The content of nonvolatile oil or organic solvent in the composition in accordance with the invention ranges for example from 0.01 to 30% by weight, in particular from 0.1 to 25% by weight and better still from 0.1 to 20% by weight, with respect to the total weight of the composition.

The compositions in accordance with the invention can also comprise at least one colouring material, such as pulverulent materials, fat-soluble dyes or water-soluble dyes.

The pulverulent colouring materials can be chosen from pigments and pearlescent agents.

The pigments can be white or coloured, inorganic and/or organic and coated or uncoated. Mention may be made, among inorganic pigments, of titanium dioxide, optionally treated at the surface, zirconium, zinc or cerium oxides, and also iron or chromium oxides, manganese violet, ultramarine blue, chromium hydrate and ferric blue. Mention may be made, among organic pigments, of carbon black, pigments of D & C type, and lakes, based on cochineal carmine, of barium, strontium, calcium or aluminium.

The pearlescent agents can be chosen from white pearlescent pigments, such as mica covered with titanium oxide or with bismuth oxychloride, coloured pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

The fat-soluble dyes are, for example, Sudan red, D&C Red 17, D&C Green 6, β-carotene, soybean oil, Sudan brown, D&C Yellow 11, D&C Violet 2, D&C Orange 5, quinoline yellow or annatto.

These colouring materials can be present in a content ranging for example from 0.01 to 30% by weight, with respect to the total weight of the composition.

The compositions in accordance with the invention can also comprise at least one filler.

The fillers can be chosen from those well known to a person skilled in the art and commonly used in cosmetic compositions. The fillers can be inorganic or organic and lamellar or spherical. Mention may be made of talc, mica, silica, kaolin, powders formed of polyamide, such as Nylon®, sold under the name Orgasol® by Atochem, of poly-β-alanine and of polyethylene, powders formed of tetrafluoroethylene polymers, such as Teflon®, lauroyllysine, starch, boron nitride, hollow polymer microspheres which are expanded, such as those of poly(vinylidene chloride)/acrylonitrile, for example those sold under the name Expancel® by Nobel Industrie, acrylic powders, such as those sold under the name Polytrap® by Dow Corning, particles formed of polymethyl methacrylate and silicone resin microbeads (Tospearls® from Toshiba, for example), precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms and in particular from 12 to 18 carbon atoms, for example zinc stearate, magnesium stearate, lithium stearate, zinc laurate or magnesium myristate.

Use may also be made of a compound capable of swelling when heated and in particular of heat-expandable particles, such as nonexpanded microspheres formed of vinylidene chloride/acrylonitrile/methyl methacrylate copolymer or of copolymer of homopolymer of acrylonitrile, such as, for example, those sold respectively under the references Expancel® 820 DU 40 and Expancel® 007WU by Akzo Nobel.

The fillers can represent for example from 0.1 to 25% by weight, in particular from 0.2 to 20% by weight, with respect to the total weight of the composition.

The compositions in accordance with the invention can also comprise at least one fibre which makes possible an improvement in the lengthening effect.

The term “fibre” should be understood as meaning an object with a length L and a diameter D such that L is much greater than D, D being the diameter of the circle in which the cross section of the fibre is framed. In particular, the L/D ratio (or aspect ratio) is chosen within the range from 3.5 to 2500, in particular from 5 to 500 and more particularly from 5 to 150.

The fibres which can be used in the composition of the invention can be fibres of synthetic or natural and inorganic or organic origin. They can be short or long, individual or organized, for example plaited, and hollow or solid. They can have any shape and can in particular be circular or polygonal (square, hexagonal or octagonal) in cross section, according to the specific application envisaged. In particular, their ends are blunted and/or polished to prevent injury.

In particular, the fibres preferably have a length ranging for example from 1 μm to 10 mm, in particular from 0.1 mm to 5 mm and more particularly from 0.3 mm to 3.5 mm. Their cross section can be included within a circle with a diameter ranging for example from 2 nm to 500 μm, in particular ranging for example from 100 nm to 100 μm and more particularly ranging for example from 1 μm to 50 μm. The weight or count of the fibres is often given in denier or decitex and represents the weight in grams per 9 km of yarn. The fibres according to the invention can in particular have a count chosen within the range from 0.15 to 30 denier and in particular from 0.18 to 18 denier.

The fibres which can be used in the composition of the invention can be chosen from rigid or nonrigid fibres. They can be of synthetic or natural and inorganic or organic origin.

Furthermore, the fibres may or may not be surface treated, may or may not be coated and may or may not be coloured.

Mention may be made, as fibres which can be used in the composition according to the invention, of fibres which are not rigid, such as polyamide (Nylon®) fibres, or fibres which are rigid, such as polyimideamide fibres, for example those sold under the Kermel® or Kermel Tech® names by Rhodia, or poly(p-phenylene terephthalamide) (or aramid) fibres, sold in particular under the Kevlar® name by DuPont de Nemours.

The fibres can be present in the composition according to the invention in a content ranging for example from 0.01% to 10% by weight, with respect to the total weight of the composition, in particular from 0.1% to 5% by weight and more particularly from 0.3% to 3% by weight.

The compositions in accordance with the invention can also comprise at least one cosmetic active principle.

Mention may in particular be made, as cosmetic active principles which can be used in the compositions in accordance with the invention, of antioxidants, preservatives, fragrances, neutralizing agents, emollients, thickeners, coalescence agents, plasticizers, moisturizing agents, vitamins and screening agents, in particular sunscreens, and their mixtures.

Of course, a person skilled in the art will take care to choose the optional additional additives and/or their amounts so that the advantageous properties of the composition according to the invention are not, or not substantially, detrimentally affected by the envisaged addition.

The following examples are given by way of illustration of the present invention and cannot limit the scope thereof.

Unless otherwise indicated, the amounts shown are expressed as percentage by weight with respect to the total weight of the composition.

### EXAMPLE 1

### Mascara

The following composition was produced.

| Composition | |
|---|---|
| Beeswax | 3.45 |
| Carnauba wax | 2.73 |
| Paraffin wax | 10.91 |
| Black iron oxide | 2.95 |
| Ultramarine blue | 2.5 |
| Preservatives | 0.7 |
| Lysine | 2.07 |
| Stearic acid(*) | 7.27 |
| Hydroxyethylcellulose | 0.91 |
| Gum arabic | 3.45 |
| Mixture of polydimethylsiloxane | 0.13 |
| and of hydrated silica | |
| D-Panthenol | 0.5 |
| Demineralized water | q.s. for 100 |

(*)Stearic acid, triple-pressed; (C₁₆/C₁₈: 50/50)

The viscosity of the composition, measured according to the protocol described above, is 14 Pa·s. This mascara exhibits a satisfactory consistency and good dispersion of the solid particles. It is easily applied to the eyelashes and forms a smooth and homogeneous charging deposited layer.

### EXAMPLE 2

### Mascara

| | |
|---|---|
| Beeswax | 30% |
| Sodium palmitoyl sarcosinate | 5% |
| (Nikkol sarcosinate PN) | |
| Hydroxyethylcellulose | 0.94% |
| Antifoaming agent (simethicone) | 0.4% |
| Preservatives | q.s. |
| Water | q.s. for 100 |

### EXAMPLE 3

| | |
|---|---|
| Candelilla wax | 30% |
| Sodium palmitoyl sarcosinate | 5% |
| (Nikkol sarcosinate PN) | |
| Hydroxyethylcellulose | 0.94% |
| Antifoaming agent (simethicone) | 0.4% |
| Preservatives | q.s. |
| Water | q.s. for 100 |

### EXAMPLE 4

| | |
|---|---|
| Beeswax | 30% |
| Mixture of stearoyl sarcosine and | 1.25% |
| myristoyl sarcosine 75/25 | |
| (Crodasin SM from Croda) | |
| Potassium hydroxide | 0.86% |
| Hydroxyethylcellulose | 0.94% |
| Antifoaming agent (simethicone) | 0.4% |
| Preservatives | q.s. |
| Water | q.s. for 100 |

### EXAMPLE 5

| | |
|---|---|
| Paraffin wax | 7.5% |
| Beeswax | 11.25% |
| Candelilla wax | 11.25% |
| Sodium palmitoyl sarcosinate | 5% |
| (Nikkol sarcosinate PN) | |
| Hydroxyethylcellulose | 0.94% |
| Antifoaming agent (simethicone) | 0.4% |
| Preservatives | q.s. |
| Water | q.s. for 100 |

### EXAMPLE 6

| | |
|---|---|
| Paraffin wax | 7.5% |
| Beeswax | 11.25% |
| Candelilla wax | 11.25% |
| Sodium palmitoyl sarcosinate | 5% |
| (Nikkol sarcosinate PN) | |
| Hydroxyethylcellulose | 0.94% |
| Pigments | 7% |
| Antifoaming agent (simethicone) | 0.4% |
| Preservatives | q.s. |
| Water | q.s. for 100 |

### Rheology

The consistency of these compositions 2 to 6 was measured.

The measurements are carried out on an RS 600 controlled-stress rheometer from ThermoRheo equipped with a thermostatically controlled bath and with a stainless steel rotor of cone/plate geometry, with a diameter of 35 mm and an angle of 2°, at a frequency of 1 Hz, stress sweep between 0.01 and 1000 Pa. The 2 surfaces are “sanded” to limit phenomena of sliding at the walls.

The measurements are carried out at 25° C.±1° C.

The results obtained are as follows:

| Example | G* (Pa) |
|---|---|
| 2 | 50 |
| 3 | 120 |
| 4 | 4000 |
| 5 | 60 |
| 6 | 5000 |

### EXAMPLE 7

| | |
|---|---|
| Beeswax | 4.07% |
| Paraffin wax | 12.86% |
| Carnauba wax | 3.21% |
| Gum arabic | 3.39% |
| Hydroxyethylcellulose | 0.89% |
| Sodium palmitoyl sarcosinate | 1.66% |
| (Nikkol sarcosinate PN) | |
| Stearyl alcohol | 2.22% |
| Cetyl alcohol | 1.11% |
| Black iron oxides | 7.14% |
| Preservatives | q.s. |
| Water | q.s. for 100 |

The aqueous phase is prepared by heating at 93° C., with vigorous stirring, the water, the (co)surfactants, the gum arabic and the hydroxyethylcellulose.

The ingredients of the fatty phase (premilled iron oxides and wax(es)) are heated at 98° C. and then the fatty phase is added to the aqueous phase with vigorous stirring to produce the emulsion.

The mixture is allowed to cool to ambient temperature.

This mascara exhibits a viscosity, measured according to the protocol indicated above, of 10 Pa·s.

Conclusions: The mascaras of Examples 1 to 6 exhibit a satisfactory consistency and good dispersion of the solid particles, as desired for this type of product. These mascaras are easily applied to the eyelashes and form a smooth and homogeneous charging deposited layer.

### EXAMPLE 8

### Mascara

| | |
|---|---|
| Beeswax | 30% |
| Sodium stearoyl glutamate | 5% |
| (Amisoft HS-11P from Ajinomoto) | |
| Hydroxyethylcellulose | 0.89% |
| Antifoaming agent (simethicone) | 0.4% |
| Preservatives | q.s. |
| Water | q.s. for 100 |

### EXAMPLE 9

### Mascara

| | |
|---|---|
| Paraffin wax | 25% |
| Sodium stearoyl glutamate | 5% |
| (Amisoft HS-11P from Ajinomoto) | |
| Hydroxyethylcellulose | 0.89% |
| Pigments (iron oxide) | 5% |
| Antifoaming agent (simethicone) | 0.4% |
| Preservatives | q.s. |
| Water | q.s. for 100 |

### EXAMPLE 10

### Mascara

| | |
|---|---|
| Beeswax | 30% |
| Disodium stearoyl glutamate | 5% |
| (Amisoft HS-21P from Ajinomoto) | |
| Hydroxyethylcellulose | 0.89% |
| Antifoaming agent (simethicone) | 0.4% |
| Preservatives | q.s. |
| Water | q.s. for 100 |

### EXAMPLE 11

### Mascara

| | |
|---|---|
| Candelilla wax | 25% |
| Disodium stearoyl glutamate | 5% |
| (Amisoft HS-21P from Ajinomoto) | |
| Hydroxyethylcellulose | 0.89% |
| Pigments (iron oxide) | 5% |
| Antifoaming agent (simethicone) | 0.4% |
| Preservatives | q.s. |
| Water | q.s. for 100 |

The rigidity modulus G* of each of the compositions 8 to 11 is measured according to the protocol described above.

| Results | Example 8 | Example 9 | Example 10 | Example 11 |
|---|---|---|---|---|
| G* (in Pa) | 16 000 | 230 | 250 | 4000 |

The mascaras of Examples 8 to 11 exhibit a satisfactory consistency and good dispersion of the waxes and pigments, as desired for this type of product.

These mascaras are easily applied to the eyelashes and form a smooth and homogeneous charging deposited layer.

These mascaras are prepared according to the following procedure:
the fatty phase (wax) is heated at 98° C.,
the aqueous phase, preheated to 93° C., is added with vigorous stirring to produce the emulsion.

### EXAMPLE 12

### Mascara

| | |
|---|---|
| Beeswax | 30% |
| Sodium cocoyl glycinate | 5% |
| (Amilite GCS-12 from Ajinomoto) | |
| Hydroxyethylcellulose | 0.89% |
| Antifoaming agent (simethicone) | 0.4% |
| Preservatives | q.s. |
| Water | q.s. for 100 |

### EXAMPLE 13

### Mascara

| | |
|---|---|
| Candelilla wax | 25% |
| Sodium cocoyl glycinate | 5% |
| (Amilite GCS-12 from Ajinomoto) | |
| Hydroxyethylcellulose | 0.89% |
| Black iron oxide | 5% |
| Antifoaming agent (simethicone) | 0.4% |
| Preservatives | q.s. |
| Water | q.s. for 100 |

The rigidity modulus G* of each of the compositions is measured according to the protocol described above.

| Results | Example 12 | Example 13 |
|---|---|---|
| G* (in Pa) | 49 | 100 |

The mascaras of Examples 12 and 13 exhibit a satisfactory consistency and good dispersion of the waxes and pigments, which provides a black colour for the mascara of Example 13, as desired for this type of product.

These mascaras are easily applied to the eyelashes and form a smooth and homogeneous charging deposited layer.

The above written description of the invention provides a manner and process of making and using it such that any person skilled in this art is enabled to make and use the same, this enablement being provided in particular for the subject matter of the appended claims, which make up a part of the original description and including a cosmetic composition for coating the eyelashes comprising an aqueous phase and an emulsifying system, wherein the emulsifying system comprises at least one surfactant chosen from:
the combination of at least one C₁₆-C₃₀ fatty acid and of at least one basic amino acid,
at least one glutamic acid compound and/or one of its salts,
at least one sarcosine compound of formula:

CH₃—N(R)—CH₂—COOH
in which R is an acyl group O═CR′, R′ being a saturated or unsaturated, linear or branched, hydrocarbon chain comprising from 10 to 30 carbon atoms,
or one of its cosmetically acceptable salts,
a glycine compound and/or a salt of the compound, and their mixtures,
   the combination of C₁₆-C₃₀ fatty acid and of basic amino acid or the glutamic acid compound and/or its salt or the sarcosine compound and/or its salt or the glycine compound and/or its salt constituting the main surfactant system of the composition.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising an aqueous phase and an emulsifying system, wherein the emulsifying system comprises, as the main surfactant system of the composition, at least one surfactant chosen from:
the combination of at least one C₁₆-C₃₀ fatty acid and of at least one basic amino acid,
at least one glutamic acid compound and/or one of its salts,
at least one sarcosine compound of formula:
CH₃—N(R)—CH₂—COOH
in which R is an acyl group O═CR′, R′ being a saturated or unsaturated, linear or branched, hydrocarbon chain comprising from 10 to 30 carbon atoms,
or one of its cosmetically acceptable salts, and
a glycine compound and/or a salt thereof, and their mixtures.

2. The composition according to claim 1, comprising at least one combination of at least one C₁₆-C₃₀ fatty acid and of at least one basic amino acid, wherein the C₁₆-C₃₀ fatty acid is stearic acid.

3. The composition according to claim 1, wherein the content of fatty acid(s) ranges from 0.1 to 20% by weight with respect to the total weight of the composition.

4. The composition according to claim 1, comprising at least one combination of at least one C₁₆-C₃₀ fatty acid and of at least one basic amino acid, wherein the basic amino acid is chosen from lysine, arginine and histidine.

5. The composition according to claim 1, wherein the content of basic amino acid ranges from 0.1% to 20% by weight with respect to the total weight of the composition.

6. The composition according to claim 1, wherein the combination of at least one C₁₆-C₃₀ fatty acid and of at least one basic amino acid constitutes the main surfactant system of the composition.

7. The composition according to claim 1, comprising at least one sarcosine compound chosen from sodium palmitoyl sarcosinate, magnesium palmitoyl sarcosinate, myristoyl sarcosine, stearoyl sarcosine and their mixtures.

8. The composition according to claim 1, wherein the content of sarcosine compound(s) ranges from 0.1 to 15% by weight with respect to the total weight of the composition.

9. The composition according to claim 1, wherein the sarcosine compound constitutes the main surfactant system of the composition.

10. The composition according to claim 1, comprising at least one glutamic acid compound or its salt chosen from acyl glutamic acids, their salts and their mixtures.

11. The composition according to claim 1, comprising at least one glutamic acid compound chosen from acyl glutamic acids having an acyl group comprising from 10 to 30 carbon atoms.

12. The composition according to claim 1, comprising at least one glutamic acid compound or its salt chosen from lauroyl glutamic acid, cocoyl glutamic acid, sodium stearoyl glutamate, potassium lauroyl glutamate, potassium cocoyl glutamate, sodium olivoyl glutamate, disodium stearoyl glutamate, disodium hydrogenated tallow glutamate and their mixtures.

13. The composition according to claim 1, wherein the glutamic acid compounds or their salts are present in a content ranging from 0.1 to 20% by weight with respect to the total weight of the composition.

14. The composition according to claim 1, wherein the glutamic acid compounds or their salts constitute the main surfactant system of the composition.

15. The composition according to claim 1, comprising at least one glycine compound or its salt chosen from acyl glycinates of formula (I):
R—HNCH₂COOX
in which R represents an acyl group R′C═O, and R′ represents a saturated or unsaturated, linear or branched, hydrocarbon chain comprising from 10 to 30 carbon atoms, and
X represents a cation chosen from ions of alkali metals, ions of alkaline earth metals, ammonium groups and their mixtures.

16. The composition according to claim 15, wherein the acyl group is chosen from the lauroyl, myristoyl, behenoyl, palmitoyl, stearoyl, isostearoyl, olivoyl, cocoyl or oleoyl groups and their mixtures.

17. The composition according to claim 1, comprising at least one glycine compound or its salt chosen from glycinates of following formula (II): 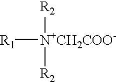
in which
R₁ represents a saturated or unsaturated, linear or branched, hydrocarbon chain comprising from 10 to 30 carbon atoms;
the R₂ groups, which are identical or different, represent an R₃OH group, R₃ being an alkyl group comprising from 2 to 10 carbon atoms.

18. The composition according to claim 17, wherein the R₁ group is chosen from lauryl, myristyl, palmityl, stearyl, cetyl, cetearyl, and oleyl groups and their mixtures.

19. The composition according to claim 1, comprising at least one glycine compound or its salt chosen from sodium cocoyl glycinate, potassium cocoyl glycinate, dihydroxyethyl oleyl glycinate, dihydroxyethyl stearyl glycinate and their mixtures.

20. The composition according to claim 1, wherein the glycine compound or its salt is present in a content ranging from 0.1 to 20% by weight with respect to the total weight of the composition.

21. The composition according to claim 1, wherein the glycine compound or its salt constitutes the main surfactant system of the composition.

22. The composition according to claim 1, wherein it comprises less than 0.5% by weight of triethanolamine stearate based on total weight.

23. The composition according to claim 1, wherein it further comprises at least one wax.

24. A method for making up or for the nontherapeutic care of the eyelashes comprising the application, to the eyelashes, of the composition according to claim 1.
